(19) United States
(12) Patent Application Publication
(10) Pub. No.: US 2018/0064953 A1
(43) Pub. Date: Mar. 8, 2018

(54) PHOTOTHERAPY DEVICE

(71) Applicant: COURTNEY VENTURES INC., Duntroon (CA)

(72) Inventors: John KENNEDY, Duntroon (CA); Simon TREADWELL, Etobicoke (CA); Roy KAYSER, Duntroon (CA)

(21) Appl. No.: 15/559,203

(22) PCT Filed: Mar. 18, 2016
(86) PCT No.: PCT/IB2016/051543

§ 371 Date: Sep. 18, 2017

Related U.S. Application Data
(60) Provisional application No. 62/134,790, filed on Mar. 18, 2015.

(51) Int. Cl. A61N 5/06

(52) U.S. Cl. CPC A61N 5/0616; A61N 2005/0652; A61N 2005/0647; A61N 2005/0626

(57) ABSTRACT

A phototherapy device includes a shell having an outer side, and an opposing inner side configured to face a treatment surface; a plurality of emitters of electromagnetic radiation that are supported by the shell and arranged to illuminate the treatment surface with the electromagnetic radiation; a holder that is connected to the shell; as well as a spacer having a head configured to adjustably connect to the holder, and a leg extending from the head and configured to abut the treatment surface for supporting the phototherapy device at an adjustable distance from the treatment surface.

### CROSS-REFERENCE TO RELATED APPLICATION

This application claims priority to U.S. provisional application No. 62/134,790, filed Mar. 18, 2015, the contents of which is incorporated herein by reference.

### FIELD

The specification relates generally to phototherapy, and specifically to a phototherapy device.

### BACKGROUND

Certain wavelengths of light (e.g. light in the red and blue parts of the visible spectrum) are known to be effective in skincare treatments (e.g. for treating acne or reducing wrinkles). Treatment devices, such as handheld lamps, are available. However, user compliance with treatment protocols is generally unsatisfactory with such devices (e.g. users do not use the devices with sufficient frequency or for a sufficient amount of time with each use), reducing treatment effectiveness.

### BRIEF DESCRIPTIONS OF THE DRAWINGS

Embodiments are described with reference to the following figures, in which:

FIG. 1 depicts an outer wall of a phototherapy device, according to a non-limiting embodiment;

FIG. 2 depicts an inner wall of the phototherapy device of FIG. 1, according to a non-limiting embodiment;

FIG. 3 depicts a support member of the phototherapy device of FIG. 1, according to a non-limiting embodiment;

FIG. 4A depicts a partial cross section of a track of the phototherapy device of FIG. 1, according to a non-limiting embodiment;

FIG. 4B depicts a partial cross section of a track of the phototherapy device of FIG. 1, according to another non-limiting embodiment;

FIG. 5 depicts an exploded view of the phototherapy device of FIG. 1, according to a non-limiting embodiment;

FIG. 6 depicts a schematic diagram of a controller of the phototherapy device of FIG. 1, according to a non-limiting embodiment; and

FIGS. 7A-7B depict alternative supports for the phototherapy device of FIG. 1, according to a non-limiting embodiment.

### DETAILED DESCRIPTION OF THE EMBODIMENTS

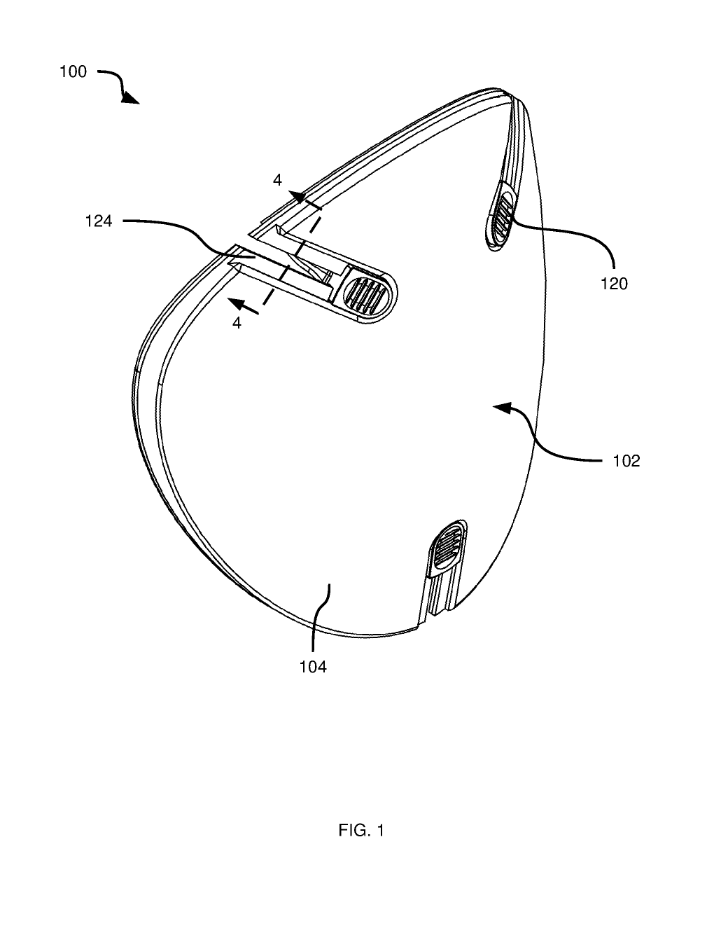

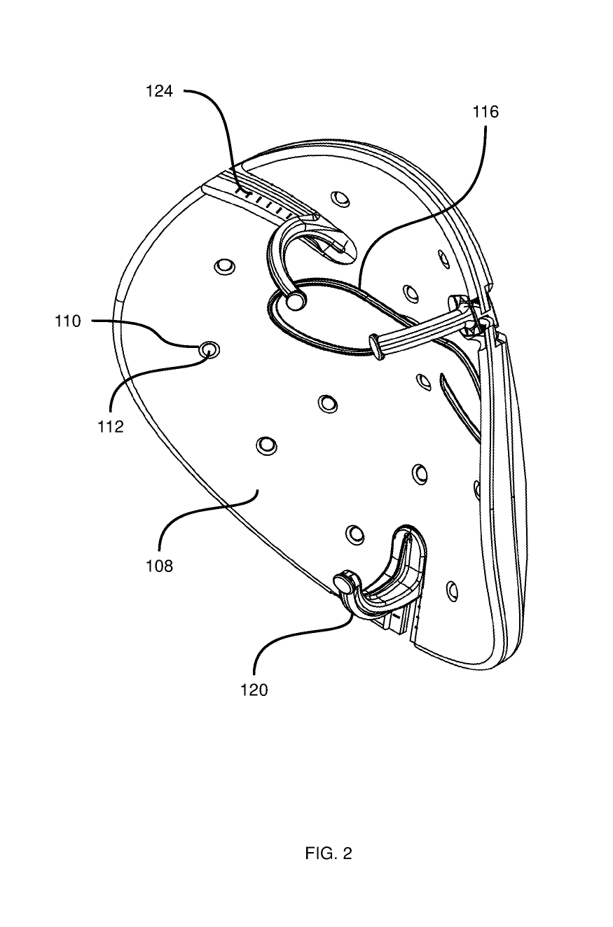

As seen in FIGS. 1 and 2, a phototherapy device 100 is shown in the form of a mask (device 100 is therefore also referred to herein as mask 100), although various other form factors for device 100 are contemplated. Device includes a shell 102 having an outer side and an opposing inner side configured to face a treatment surface (e.g. the skin of a user) when in use. In the present embodiment, shell 102 includes an outer wall 104 that defines the outer side, and thus faces away from patient tissue (e.g. skin) when mask 100 is in use. Shell 102 also includes an inner wall 108 that defines the opposing inner side, and thus faces towards patient tissue when mask 100 is in use. Walls 104 and 108 can be constructed of any suitable material, and in the present embodiment are constructed of a rigid plastic. Other materials, including composites (e.g. carbon fibre-based composites), metals (e.g. aluminum), and combinations thereof can also be employed for the fabrication of walls 104 and 108. In the present embodiment, walls 104 and 108 are solid and opaque. However, in other embodiments some or all of walls 104 and 108 can be translucent or transparent. Preferably, inner wall 108 is reflective. As seen in FIGS. 1 and 2, mask 100 is shaped to be received over a user's face. However, other embodiments of device 100 may also be applied to other body parts, and may therefore be shaped accordingly.

Device 100 also includes a plurality of emitters of electromagnetic radiation supported by shell 102 and arranged to illuminate the treatment surface when device 100 is in use. Referring to FIG. 2, inner wall 108 includes a plurality of apertures 110 through which a plurality of the above-mentioned emitters, also referred to as light sources 112, such as light-emitting diodes (LEDs) protrude. Light sources 112 are supported between walls 104 and 108 by any suitable structure. Light sources 112 can emit light at any suitable combination of wavelengths. For example, light sources 112 can emit light in the blue region of the visible spectrum. In other embodiments light sources 112 can emit light in the red region of the visible spectrum. In still other embodiments, light sources 112 can emit light in the near-ultraviolet or near-infrared regions of the spectrum, or in combinations of any of the above. Further, as seen in FIG. 2, mask 100 can include an eye shield wall 116 extending outwards from inner wall 108 (that is, towards the treatment surface of the user when mask 100 is in use). In other embodiments, shield wall 116 can be omitted.

Device 100 also includes at least one holder connected to shell 102, and at least one spacer configured to adjustable connect to the holder. The spacer, as will be seen below, is configured to abut the treatment surface when device 100 is in use and thus support device 100 at an adjustable distance from the treatment surface. The distance is adjustable by virtue of the adjustable connection between the spacer and the holder.

As seen in both FIGS. 1 and 2, mask 100 includes at least one adjustable spacer 120, also referred to herein as a support member 120. Support members 120 are adjustably (specifically, slideably in the present embodiment) mounted in at least one respective holder in the form of a track 124 defined by inner and outer walls 104 and 108. In the present embodiment, three tracks 124 and three corresponding support members 120 are provided. However, in other embodiments, different numbers of tracks 124 and support members 120 may be provided.

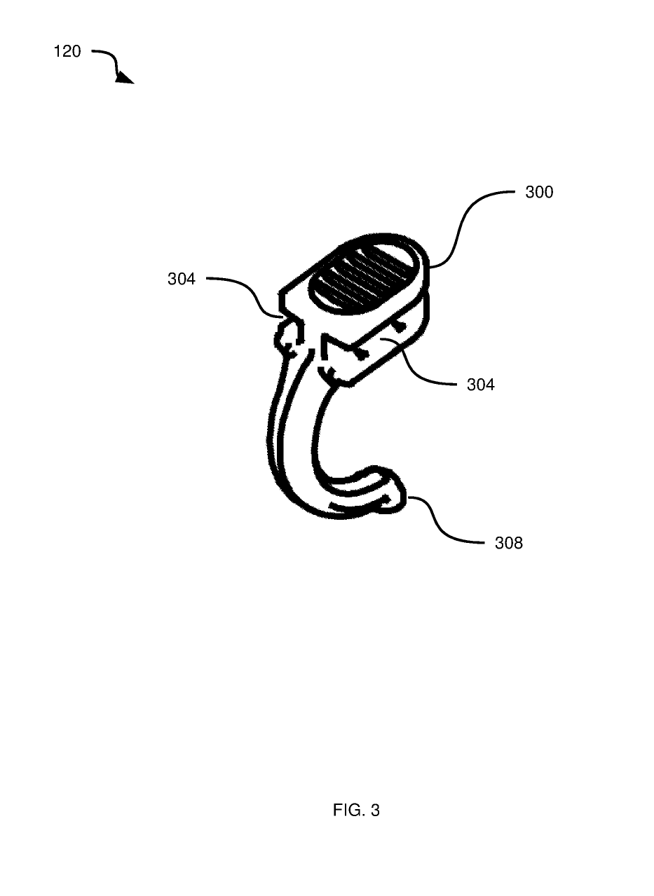

A support member 120 is shown in greater detail in FIG. 3. In particular, support member 120 includes a head 300 for engaging a corresponding track 124 on mask 100. For example, head 300 may include grooves 304 on either side thereof for receiving rails of a track 124 (to be described below in greater detail) therein and sliding along the length of the track 124. The fit between grooves 304 and the corresponding track 124 is sufficiently stiff that support member 120 is prevented from sliding along track 124 solely under the influence of its own weight or the orientation of device 100. That is, a force is applied to support member 120 (e.g. by a user of device 100) to move support member 120 within track 124.

Support member 120 also includes a leg 308 depending from head 300, for abutting a user's skin during use. Thus, the set of support members 120 shown in FIGS. 1 and 2 prevent inner wall 108 from contacting the user's skin during use, and instead maintain a predetermined distance between inner wall 108 and the user. Leg 308 may have any suitable length (e.g. based on the desired distance between mask 100 and the surface to be treated). In addition, while leg 308 is shown as being curved in the present embodiment, in other embodiments leg 308 can be straight, or have any other suitable shape. When multiple legs 308 are included with mask 100, each leg 308 can have a different shape, or the same shape as the other legs 308. Support members 120 can be moved along tracks 124 independently to adjust the distance between mask 100 and the user when in use, to accommodate the shape or size of the portion of the user being treated, or both. Support members 120 and can also be removed from tracks 124 entirely (e.g. for cleaning, packaging or replacement).

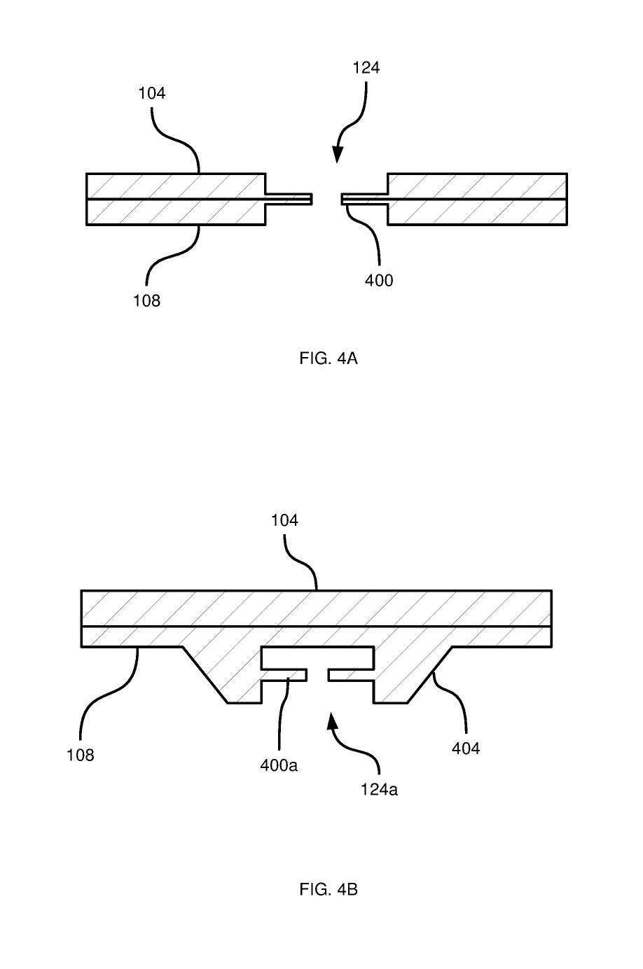

A variety of other holders are also contemplated. For example, instead of tracks 124, a plurality of snaps or other fasteners (to which support members 120 may be connected and removed, such as hook and loop fasteners) can be employed as a holder, and a support member 120 can be adjusted relative to shell 102 by selecting a subset of the fasteners to connect the support member 120 to. In addition, various configurations of tracks 124 are contemplated. Referring to FIG. 4A, a partial cross-section of device 100 is shown, taken along line 4-4 in FIG. 1 and illustrating the structure of a track 124. In particular, track 124 is formed as a slot through both outer and inner walls 104 and 108, open at the edge of shell 102. Two rails 400 extend into the slot, each rail 400 being formed by cooperating rail portions of walls 104 and 108.

FIG. 4B illustrates a variation of track 124, in the form of a track 124a. Track 124a includes rails 400a configured for receipt within grooves 304. However, rather than being defined as a slot extending through shell 102, track 124a is defined entirely by inner wall 108, for example in a boss 404 of material raised from inner wall 108.

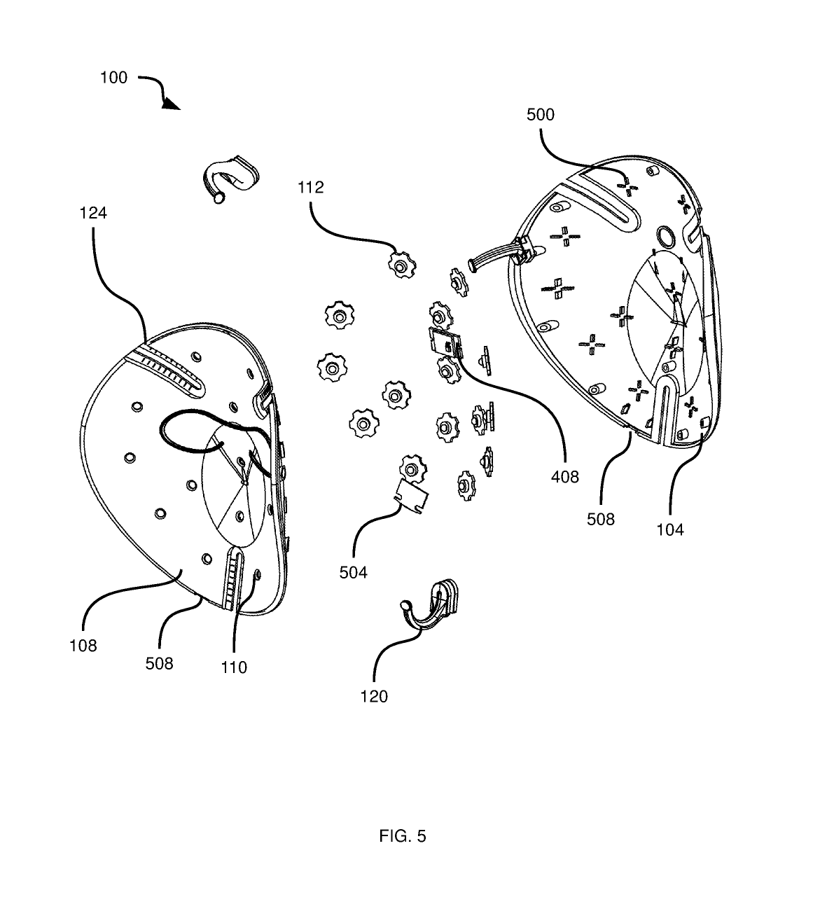

Referring now to FIG. 5, an exploded view of mask 100 is shown. As seen on the inner side of outer wall 104, mounting elements 500 are provided for light sources 112. However, it is not necessary for light sources 112 to be mounted to outer wall 104. In some embodiments, light sources 112 can be supported solely by inner wall 108, or by both inner wall 108 and outer wall 104. In addition to components discussed above, mask 100 includes connections (e.g. wiring, not shown) to provide power to light sources 112. The connections can connect light sources 112, and any other components requiring electrical power, to a power board 504, which can in turn be connected to a power source such as a battery (internal or external to mask 100) or an electrical outlet. Board 504, in addition to providing a connection to an external power source in certain embodiments, can also provide a connection to an external computing device, as will be discussed below. When the above-mentioned connections are wired, board 504 can include a port accessible via a dock 508 defined in shell 102

Mask 100 can also include a sensor board 508 including, for example, a Hall effect sensor. In some embodiments, mask 100 can be employed in conjunction with a separate eye cover (not shown) for reducing or eliminating light incident on a user's eyes when mask 100 is in use. The eye cover can include a magnetic element (e.g. between the eye-pieces), and sensor board 508 can be configured to generate a signal that prevents operation of mask 100 if the magnetic element is not detected (i.e. if the user is not wearing the eye cover). In other embodiments, sensor board 508 can be omitted. In other embodiments, a variety of other sensors can be incorporated in sensor board 508 instead of, or in addition to, the above-mentioned Hall effect sensor. The eye cover can include any suitable element corresponding to the sensors implemented on sensor board 508. For example, sensor board 508 can include an optical sensor for detecting light (either emitted by the eye cover, or the absence of which indicates the presence of the eye cover). In further embodiments, sensor board 508 can include a capacitive sensor and the eye cover can include a conductive element for contacting the sensor. In still further embodiments, sensor board 508 can include a mechanical switch and the eye cover can include an element protruding therefore for activating the switch.

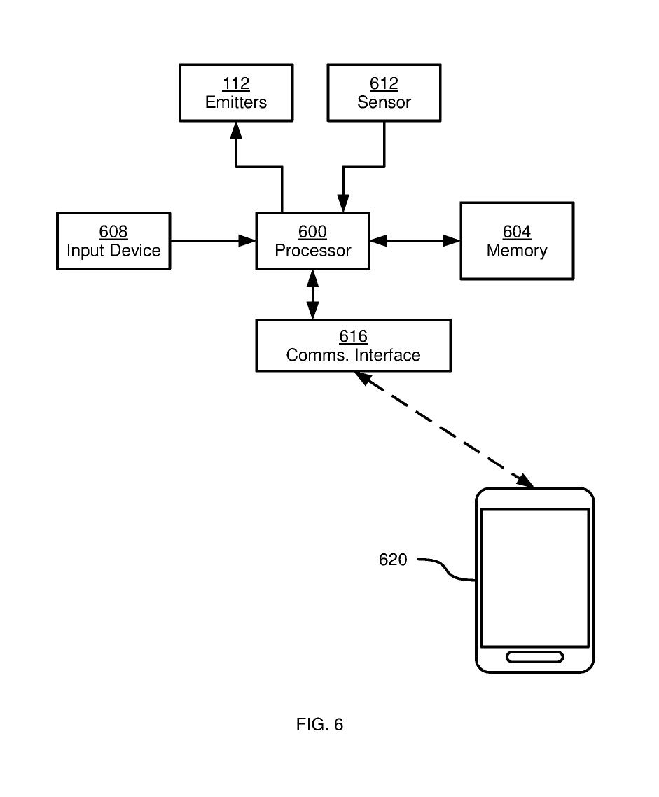

Turning to FIG. 6, Mask 100 can also include a controller, such as a microcomputer including a processor 600 and a memory 604, as well as an input device 608 (e.g. one or more buttons, switches or the like). Processor 600 and memory 604 comprise one or more integrated circuits; in general, memory 604 stores computer readable instructions for execution by processor 600. Processor 600, upon execution of the above-mentioned instructions, is configured to control the operation of device 100. Processor 600 may also respond to input data from input device 608 during such execution. For example, actuation of input device 608 may cause processor 600 to initiate the performance of a treatment protocol stored in memory 604 (e.g. to activate emitters 112 for a predetermined time period).

Processor 600 is connected with emitters 112, and may also be connected to a sensor 612 (such as the above-mentioned Hall effect sensor), when such a sensor is provided. In some embodiments, only one of sensor 612 and input device 608 may be required; that is, in the presence of sensor 612 which is configured to signal to processor 600 when a user's eye-mask or other accessory is detected, input from input device 608 may not be required to initiate operation of device 100.

Processor 600 is configured to switch light sources 112 on and off, and can also be configured to control a brightness level of light sources 112 (either as a whole, in subgroups or individually). Processor 600 can also implement a timer, for automatically switching light sources 112 on or off (or, as mentioned above, dimming or brightening light sources 112) after a predefined period of time. Device 100 can also include a network interface coupled to the controller, to enable the controller to receive control instructions from an external device 620 (e.g. a switch, a computing device such as a smart phone, and the like). Via interface 616, the controller can also transmit data to the external device. For example, external device 620 can be a smartphone that is configured, by execution of an application by its central processing unit (CPU), to receive and store data defining past treatments conducted with mask 100. For example, the smartphone can store the time and length of such treatments, and can also control treatments by sending signals to the controller of mask 100.

The above-mentioned smartphone can also be configured to generate notifications or other messages (such as a notification presented on a display of the smartphone) based on data received from the controller of mask 100. For example, the smartphone can be configured to generate reminders for future treatments, or to generate order messages for associated products (such as topical creams) based on the number and timing of treatments that have been conducted with mask 100.

With support members 120 engaged in tracks 124 as shown in FIGS. 1 and 2, mask 100 can be used by resting mask 100 on a surface of the user (such as the user's face, arm, or the like). For example, the user can lie down and rest mask 100 on their face; support members 120 support mask 100 at a predefined distance above the user's face. Other arrangements are also contemplated, however.

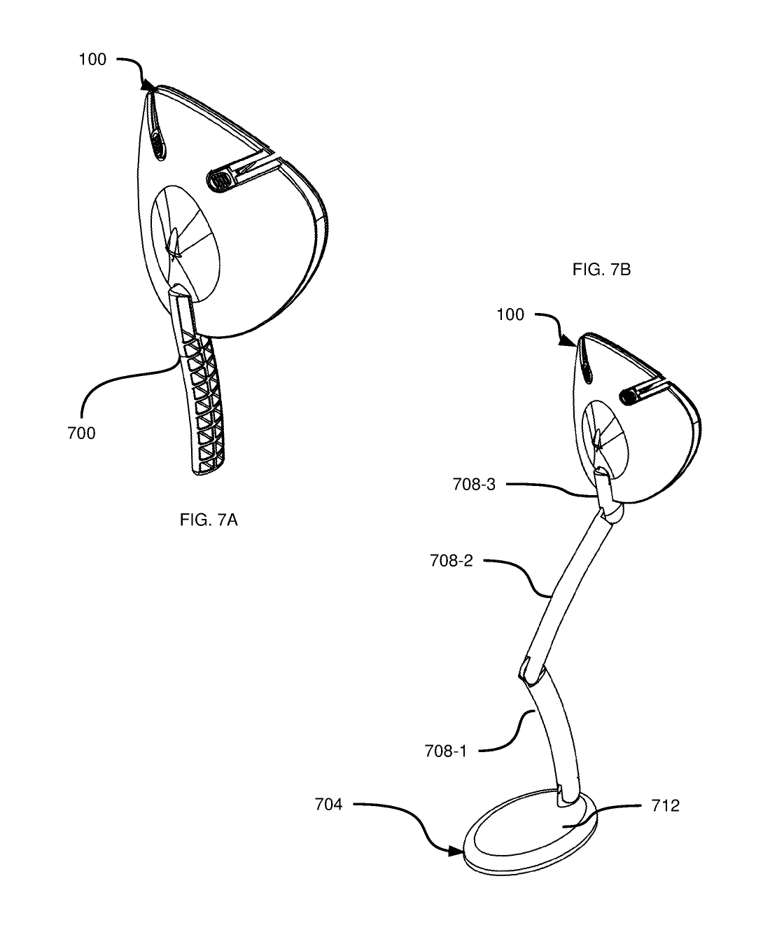

For example, turning now to FIGS. 7A and 7B, one or more support members 120 can be removed from tracks 124 and replaced with alternative supports, such as a handle 700 (FIG. 5A) or a stand 704 (FIG. 5B) including one or more articulating segments 704 (three segments 704-1, 704-2 and 704-3 are shown) supported by a base 712. Handle 700 and stand 704 (specifically, segment 708-3) can have ends adapted to fit into tracks 124. The remaining support members 120 can be removed, or (as seen in FIGS. 7A and 7B) can be retained in their respective tracks 124. In other embodiments, handle 700 and segment 704-3 of stand 704 can also each include legs similar to leg 308, for maintaining spacing between mask 100 and the user when in use. The position of mask 100 on the end of handle 700 of stand 704 is also adjustable, as handle 700 and segment 708-3 of stand 504 are slideable within tracks 124 (similarly to support members 120). A power source (such as a battery) can be contained within handle 500 or stand 504, for connection to board 504 shown in FIG. 5. In some embodiments, in addition, handle 500 can be configured to connect to stand 704 (e.g. to segment 708-2), such that segment 708-3 of stand 704 as seen in FIG. 7B is in actuality handle 700.

Variations to the above are also contemplated. For example, sensor board 408 mentioned above can be configured to detect a plurality of different magnetic signatures. Rather than simply preventing the operation of mask 100 in the absence of eye covers, sensor board 508 can be configured to distinguish between a plurality of magnetic elements, and mask 100 can be controlled (either by sensor board 508, which can carry the above-mentioned controller, or by the above-mentioned controller where the controller is supported separately from sensor board 508 based on the output of sensor board 508) to implement different treatment protocols stored in memory 604 (by turning light sources 112 on for different periods of time, different pulsing patterns, and the like) based on which magnetic element was detected. For example, different magnetic elements can be embedded in eye covers (to initiate a facial treatment protocol), necklaces (to initiate a décolletage treatment protocol), rings (to initiate a hand treatment protocol), and the like.

The scope of the claims should not be limited by the embodiments set forth in the above examples, but should be given the broadest interpretation consistent with the description as a whole.

We claim:

1. A phototherapy device, comprising:
a shell having an outer side, and an opposing inner side configured to face a treatment surface;
a plurality of emitters of electromagnetic radiation supported by the shell and arranged to illuminate the treatment surface with the electromagnetic radiation;
a holder connected to the shell; and
a spacer having a head configured to adjustably connect to the holder, and a leg extending from the head and configured to abut the treatment surface for supporting the phototherapy device at an adjustable distance from the treatment surface.

2. The phototherapy device of claim 1, further comprising:
an inner wall defining the inner side of the shell; and
an outer wall defining the outer side of the shell;
wherein the plurality of emitters are supported between the inner and outer walls.

3. The phototherapy device of claim 2, the inner wall having a plurality of apertures therethrough for permitting passage of the electromagnetic radiation.

4. The phototherapy device of claim 3, the inner wall having a concave shape for accommodating the treatment surface.

5. The phototherapy device of claim 1, wherein the shell is a mask.

6. The phototherapy device of claim 1, wherein the holder comprises a track having a rail; and
wherein the head of the spacer includes a groove for slideably receiving the rail.

7. The phototherapy device of claim 6, wherein the track includes two opposing rails; and
wherein the head of the spacer includes two opposing grooves for slidably receiving respective ones of the pair of rails.

8. The phototherapy device of claim 6, wherein the track is defined by a slot extending through the shell.

9. The phototherapy device of claim 6, wherein the track is defined by a boss on the inner wall of the shell.

10. The phototherapy mask of claim 1, further comprising:
a plurality of holders, and a corresponding plurality of independently adjustable spacers adjustably connected to the holders.
